(19) United States
(12) Patent Application Publication
(10) Pub. No.: US 2023/0190603 A1
(43) Pub. Date: Jun. 22, 2023

(54) Oral Care Compositions

(71) Applicant: Colgate-Palmolive Company, New York, NY (US)

(72) Inventors: Ruth HINRICHS, Therwil (CH); Turan MATUR, Binningen (CH); Norbert HUBER, Basel (CH); Thomas SCHOLLBACH, Basel (CH)

(73) Assignee: Colgate-Palmolive Company, New York, NY (US)

(21) Appl. No.: 17/557,317

(22) Filed: Dec. 21, 2021

(51) Int. Cl. A61K 8/365; A61K 8/19; A61K 8/21; A61K 8/44; A61K 8/49; A61Q 11/00

(52) U.S. Cl. CPC A61K 8/365; A61K 8/19; A61K 8/21; A61K 8/44; A61K 8/49; A61Q 11/00

(57) ABSTRACT

Described herein are oral care compositions, and in particular to oral care mouth rinse compositions comprising a single source of stannous, wherein the single source of stannous is stannous oxalate; a tertiary amine fluoride; and at least one of a monocarboxylic acid, dicarboxylic, or a tricarboxylic acid, as well as to methods of using and of making these compositions. The present compositions are formulated to present stable, non-turbid compositions and are useful for preventing and treating diseases or conditions of the oral cavity.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Bacteria are the main cause for gingival inflammations. Therefore, antibacterial efficacy is recognized as a prerequisite for chemical treatment of irritated gums and protection against gingival inflammations.

Oral care compositions which contain stannous ion sources exhibit excellent clinical benefits, particularly in the reduction of gingivitis and in the treatment or prevention of erosive tooth demineralization. Stannous fluoride is well known for use in clinical dentistry with a history of therapeutic benefits over forty years. However, until recently, its popularity has been limited by its instability in aqueous solutions. The instability of stannous fluoride in water is primarily due to the reactivity of the stannous ion (Sn²⁺). Stannous salts readily hydrolyze above a pH of 4, resulting in precipitation from solution, with a consequent loss of the therapeutic properties.

Unfortunately, adjustments in formulation may lead to formation of turbid mouth rinses containing sediments which demonstrate limited galenic stability.

It would therefore be desirable to improve efficacy and stability of such formulations without the formation of turbidity and sedimentation.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have surprisingly discovered oral care compositions comprising stannous oxalate, a tertiary amine fluoride, and at least one of a monocarboxylic acid, dicarboxylic, or a tricarboxylic acid was both stable and efficacious. Such formulations exhibited good stannous availability and little to no turbidity. In one aspect, such compositions may be used to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health.

In at least one embodiment, the present invention is directed to an oral care composition comprising a single source of stannous, wherein the single source of stannous is stannous oxalate; a tertiary amine fluoride; and at least one of a monocarboxylic acid, dicarboxylic, or a tricarboxylic acid.

In further embodiments, the invention is directed to an oral care composition comprising a single source of stannous, wherein the single source of stannous is stannous oxalate; a tertiary amine fluoride; and at least one of a monocarboxylic acid, dicarboxylic, or a tricarboxylic acid. In certain embodiments, the oral care composition is a mouth rinse. In certain embodiments, the stannous oxalate is present at about 0.04% to about 3.0%, based on the weight of the oral care composition. In certain embodiments, the tertiary amine fluoride is present in an amount from about 0.7% to about 8.0% by weight, of the oral care composition. In certain embodiments, the tertiary amine fluoride is olaflur. In certain embodiments, the monocarboxylic acid, dicarboxylic, or a tricarboxylic acid is selected from gluconic acid, malic acid, citric acid, or combinations thereof. In certain embodiments, the total amount of monocarboxylic acid, dicarboxylic, and tricarboxylic acid present is in an amount from about 0.1% to about 5.0% by weight, of the oral care composition.

In certain embodiments, the composition further comprises amphoteric surfactant. In certain embodiments, the amphoteric surfactant is cocamidopropyl betaine. In certain embodiments, the amphoteric surfactant is present in an amount from about 2.0% to about 6.0% by weight, of the oral care composition.

In certain embodiments, the composition further comprises hydroxyethyl cellulose. In certain embodiments, the hydroxyethyl cellulose is present in an amount from about 1.0% to about 5.0% by weight, of the oral care composition.

In certain embodiments, the composition further comprises sugar alcohol. In certain embodiments, the sugar alcohol is sorbitol, xylitol, or a combination thereof. In certain embodiments, the sugar alcohol is present in an amount from about 0.5% to about 45.0% by weight, of the oral care composition.

In certain embodiments, the composition further comprises saccharin. In certain embodiments, the saccharin is present in an amount from about 0.2% to about 1.0% by weight, of the oral care composition.

In further embodiments, the invention is directed to a use of any of the compositions described herein to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health.

In other embodiments, the invention is a mouth rinse composition comprising a single source of stannous, wherein the single source of stannous is stannous oxalate; a tertiary amine fluoride; hydroxyethylcellulose; an amphoteric surfactant; and at least one of a monocarboxylic acid, dicarboxylic, or a tricarboxylic acid. In certain embodiments, the stannous oxalate is present at about 0.04% to about 3.0%, based on the weight of the mouth rinse composition. In certain embodiments, the tertiary amine fluoride is present in an amount from about 0.7% to about 8.0% by weight, of the mouth rinse composition. In certain embodiments, the tertiary amine fluoride is olaflur. In certain embodiments, the monocarboxylic acid, dicarboxylic, or a tricarboxylic acid is selected from gluconic acid, malic acid, citric acid, or combinations thereof. In certain embodiments, the monocarboxylic acid, dicarboxylic, or a tricarboxylic acid is present in an amount from about 0.3% to about 5.0% by weight, of the mouth rinse composition. In certain embodiments, the amphoteric surfactant is cocamidopropyl betaine. In certain embodiments, the amphoteric surfactant is present in an amount from about 2.0% to about 6.0% by weight, of the mouth rinse composition. In certain embodiments, the hydroxyethyl cellulose is present in an amount from about 1.0% to about 5.0% by weight, of the mouth rinse composition. In certain embodiments, the composition further comprises sugar alcohol. In certain embodiments, the sugar alcohol is sorbitol, xylitol, or a combination thereof. In certain embodiments, the sugar alcohol is present in an amount from about 0.5% to about 45.0% by weight, of the mouth rinse composition. In certain embodiments, the composition further comprises saccharin. In certain embodiments, the saccharin is present in an amount from about 0.2% to about 1.0% by weight, of the mouth rinse composition.

In further embodiments, the invention is directed to a method of treatment or prevention of erosive tooth demineralization, gingivitis, plaque, and/or dental caries, the method comprising the application to the oral cavity of a person in need thereof an oral care composition comprising a single source of stannous, wherein the single source of stannous is stannous oxalate; a tertiary amine fluoride; and at least one of a monocarboxylic acid, a dicarboxylic, or a tricarboxylic acid.

Further areas of applicability of the present invention will become apparent from the detailed description provided hereinafter. It should be understood that the detailed description and specific examples, while indicating the typical embodiments of the invention, are intended for purposes of illustration only and are not intended to limit the scope of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition.

According to the present application, use of the term “about” in conjunction with a numeral value refers to a value that may be +/- 5% of that numeral. As used herein, the term “substantially free” is intended to mean an amount less than about 5.0 weight %, less than 3.0 weight %, 1.0 wt.%; preferably less than about 0.5 wt.%, and more preferably less than about 0.25 wt.% of the composition.

As used herein, the term “effective amount” refers to an amount that is effective to elicit the desired biological response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, i.e. , a single dose or multiple doses may be required to achieve the desired endpoint.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present disclosure is directed toward oral care compositions and methods of using such oral care compositions for the treatment or prevention of erosive tooth demineralization, gingivitis, plaque, and/or dental caries. In certain embodiments, the oral care composition is a mouth rinse.

The present inventors have surprisingly and unexpectedly discovered that providing an oral care composition comprising stannous oxalate, a tertiary amine fluoride, and at least one of a monocarboxylic acid, dicarboxylic, or a tricarboxylic acid provides for stable and efficacious formulations.

The stannous oxalate may be present at various amounts or concentrations. In one embodiment, stannous oxalate may be present in an amount of from about 0.04% to about 3.0%, based on the weight of the oral care composition. For example, stannous oxalate may be present in an amount of about 0.04 weight %, about 0.05 weight %, about 0.07 weight %, about 0.1 weight %, about 1.2 weight %, about 1.5 weight %, about 1.8 weight %, about 2.0 weight %, about 2.5 weight %, or about 3.0 weight %. In another example, stannous oxalate may be present in an amount of from about 0.04% to about 2.5%, about 0.04% to about 2.0%, about 0.04% to about 1.5%, or about 0.04% to about 1%, based on the weight of the oral care composition. In further embodiments, stannous oxalate is present in an amount of about 0.04% or more, about 0.05% or more, about 0.07% or more, about 0.1% or more, about 0.5% or more, about 0.6% or more, or about 0.7% or more up to about 3.0%, based on the weight of the oral care composition. In further embodiments, stannous oxalate is present in an amount of about 0.04% or more, about 0.05% or more, about 0.07% or more, about 0.1% or more, about 0.5% or more, about 0.6% or more, or about 0.7% or more up to about 1.0%, based on the weight of the oral care composition. In further embodiments, stannous oxalate is present in an amount of about 0.04% to about 4%, about 0.04% to about 3.0%, about 0.05% to about 2.5%, or about 0.05% to about 1.5%, based on the weight of the oral care composition.

In certain embodiments, stannous is present in the composition in an amount to yield from about 3000 ppm to about 9500 ppm. For example, stannous may be present in an amount of about 3000 ppm, about 3500 ppm, about 4000 ppm, about 4500 ppm, about 5000 ppm, about 5500 ppm, about 6000 ppm, about 6500 ppm, about 7000 ppm, about 7500 ppm, about 8000 ppm, about 8500 ppm, about 9000 ppm, or about 9500 ppm. In another example, stannous may be present in an amount of from about 3000 ppm to about 9000 ppm, about 4000 ppm to about 9000 ppm, about 5000 ppm to about 9000 ppm, or about 6000 ppm to about 9000 ppm. In further embodiments, stannous is present in an amount of about 3000 ppm or more, about 4000 ppm or more, about 5000 ppm or more, about 6000 ppm or more up to about 9500 ppm. In further embodiments, stannous is present in an amount of about 3000 ppm or more, about 4000 ppm or more, about 5000 ppm or more, about 6000 ppm or more, about 7000 ppm or more, or about 8000 ppm or more up to about 9000 ppm. In further embodiments, stannous is present in an amount of about 4000 ppm to about 9500 ppm, about 5000 ppm to about 9000 ppm, about 6000 ppm to about 9000 ppm, or about 6500 ppm to about 9000 ppm.

One preferred tertiary amine fluoride is the compound N,N,N′-tris(2-hydroxyethyl)-N′-octadecyl-1,3-diaminopropane dihydrofluoride (also known as “Olaflur”, sometimes written as N,N,N′-tris(2-hydroxyethyl)-N′-octadecyl propane-1,3-diamine dihydrofluoride, and corresponds to CAS Registry number 6818-37-7). However, one of skill in the art would appreciate that any of a variety of tertiary amine fluorides are known to be useful in mouth rise compositions can be employed instead of, or in addition to, N,N,N′-tris(2-hydroxyethyl)-N′-octadecyl-1,3-diaminopropane dihydrofluoride.

The tertiary amine fluoride may be present at various amounts or concentrations. In one embodiment, the tertiary amine fluoride may be present in an amount of from about 0.7% to about 8%, based on the weight of the oral care composition. For example, the tertiary amine fluoride may be present in an amount of about 0.7 weight %, about 0.9 weight %, about 1.1 weight %, about 1.3 weight %, about 1.5 weight %, about 1.8 weight %, about 2.0 weight %, about 2.2 weight %, about 2.6 weight %, about 3.0 weight %, about 3.5 weight %, about 4.0 weight %, about 4.5 weight %, about 5.0 weight %, about 5.2 weight %, about 5.5 weight %, about 6.0 weight %, about 6.5 weight %, about 7.0 weight %, about 7.5 weight %, or about 8.0 weight %. In another example, the tertiary amine fluoride may be present in an amount of from about 0.7% to about 2.0%, about 0.8% to about 2.0%, about 1.0% to about 2.0%, or about 0.9% to about 1.5%, based on the weight of the oral care composition. In further embodiments, the tertiary amine fluoride is present in an amount of about 0.7% or more, about 0.9% or more, about 1.3% or more, or about 1.8% or more up to about 6%, based on the weight of the oral care composition. In further embodiments, the tertiary amine fluoride is present in an amount of about 0.7% to 7%, about 0.8% to about 6%, about 0.8% to about 5.5%, or about 0.8% to about 5.2%, based on the weight of the oral care composition.

In certain embodiments, the tertiary amine fluoride is present in the composition in an amount to yield from about 500 ppm to about 2500 ppm. For example, the tertiary amine fluoride may be present in an amount of about 1000 ppm, about 1500 ppm, about 2000 ppm, or about 2500 ppm. In another embodiments, the tertiary amine fluoride may be present in an amount of from about 1000 ppm to about 2000 ppm, about 1000 ppm to about 1500 ppm, or about 1000 ppm to about 1400 ppm. In further embodiments, the tertiary amine fluoride is present in an amount of about 500 ppm or more, about 1000 ppm or more, about 1200 ppm or more, about 1400 ppm or more up to about 1500 ppm. In further embodiments, the tertiary amine fluoride is present in an amount of about 500 ppm or more, about 1000 ppm or more up to about 2000 ppm. In further embodiments, the tertiary amine fluoride is present in an amount of about 500 ppm to about 2500 ppm, about 500 ppm to about 2000 ppm, about 1000 ppm to about 2000 ppm, or about 500 ppm to about 1500 ppm.

The monocarboxylic acid may be any monocarboxylic acid suitable for oral care compositions. In preferred embodiments, the monocarboxylic acid is gluconic acid. The dicarboxylic acid may be any dicarboxylic acid suitable for oral care compositions. In preferred embodiments, the dicarboxylic acid may be malic acid, succinic acid, tartaric acid, or a combination thereof. The tricarboxylic acid may be any tricarboxylic acid suitable for oral care compositions. In preferred embodiments, the tricarboxylic acid is citric acid.

The monocarboxylic acid, dicarboxylic, and/or tricarboxylic acid may be present at various amounts or concentrations. In certain embodiments, the monocarboxylic acid, dicarboxylic, and/or tricarboxylic acid may be included in various forms, such as but not limited to, a free base, a salt such as a hydrochloride (HCl) salt, or peptidyl forms such as, but not limited to, mono-, di-, tri-, and oligo-peptide forms. In one embodiment, the monocarboxylic acid, dicarboxylic, and/or tricarboxylic acid may be present in a total amount of from about 0.1% to about 6%, based on the weight of the oral care composition. For example, monocarboxylic acid, dicarboxylic, and/or tricarboxylic acid may be present in a total amount of about 0.1 weight %, 0.3 weight %, about 0.5 weight %, about 0.7 weight %, about 1.0 weight %, about 1.3 weight %, about 1.6 weight %, about 2.0 weight %, about 2.5 weight %, about 3.0 weight %, about 3.5 weight %, about 4.0 weight %, about 4.5 weight %, about 5.0 weight %, about 5.5 weight %, or about 6.0 weight %. In another example, the monocarboxylic acid, dicarboxylic, and/or tricarboxylic acid may be present in a total amount of from about 0.1% to about 6%, about 1.0% to about 6.0%, about 2.0% to about 5.0%, or about 4.0% to about 6.0%, based on the weight of the oral care composition. In further embodiments, the monocarboxylic acid, dicarboxylic, and/or tricarboxylic acid may be present in a total amount of about 0.1% or more, about 0.5% or more, about 0.8% or more, or about 1% or more up to about 5%, based on the weight of the oral care composition. In further embodiments, the monocarboxylic acid, dicarboxylic, and/or tricarboxylic acid may be present in a total amount of about 0.1% to 6%, about 0.5% to about 5%, about 1% to about 6%, or about 2% to about 5%, based on the weight of the oral care composition.

Amphoteric surfactant is useful for compositions described herein. The amphoteric surfactant may be present at various amounts or concentrations. In certain embodiments, the amphoteric surfactant is selected from cocamidopropyl betaine, lauramidopropyl betaine, cocobetaine, cocamidopropyl hydroxysultaine, and combinations thereof. In certain preferred embodiments, the amphoteric surfactant is cocamidopropyl betaine. The oral care composition may comprise the amphoteric surfactant at various amounts or concentrations. In certain embodiments, the amphoteric surfactant may be present in an amount from about 2% to about 6%, from about 2% to about 5%; or from about 3% to about 5%, based on the weight of the oral care composition. In certain embodiments, the amphoteric surfactant may be present at about 4%, at about 4.5%, at about 5%, or at about 5.5%, based on the weight of the oral care composition.

Hydroxyethyl celluloses are well known in the art. The hydroxyethyl cellulose may be present at various amounts or concentrations. In one embodiment, the hydroxyethyl cellulose may be present in an amount of from about 1% to about 5%, based on the weight of the oral care composition. For example, the hydroxyethyl cellulose may be present in an amount of about 1.0 weight %, about 1.3 weight %, about 1.6 weight %, about 2.0 weight %, about 2.2 weight %, about 2.6 weight %, about 3.0 weight %, about 3.3 weight %, about 3.6 weight %, about 4.0 weight %, about 4.3 weight %, about 4.6 weight %, or about 5.0 weight %. In another example, the hydroxyethyl cellulose may be present in an amount of from about 1% to about 4%, about 1.5% to about 4.0%, about 1.8% to about 4.5%, or about 2% to about 3.5%, based on the weight of the oral care composition. In further embodiments, the hydroxyethyl cellulose is present in an amount of about 1.0% or more, about 1.5% or more, about 1.8% or more, or about 2.2% or more up to about 5.0%, based on the weight of the oral care composition. In further embodiments, the hydroxyethyl cellulose is present in an amount of about 1.0% to 5.5%, about 1.5% to about 5.0%, about 1.5% to about 4.0%, about 1.5% to about 3.5%, or about 2% to about 3%, based on the weight of the oral care composition.

Sugar alcohols are well known in the art. In certain embodiments, the sugar alcohol is selected from sorbitol, xylitol, or a combination thereof. The sugar alcohol may be present at various amounts or concentrations. In one embodiment, the sugar alcohol may be present in an amount of from about 0.5% to about 45.0%, based on the weight of the oral care composition. For example, the sugar alcohol may be present in an amount of about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, about 5.0 weight %, about 6.0 weight %, about 10.0 weight %, about 15.0 weight %, about 20.0 weight %, about 25.0 weight %, about 30.0 weight %, about 35.0 weight %, about 40.0 weight %, or about 45 weight %. In another example, the sugar alcohol may be present in an amount of from about 0.5% to about 40%, about 5% to about 40%, about 10% to about 40%, or about 20% to about 40%, based on the weight of the oral care composition. In further embodiments, the sugar alcohol is present in an amount of about 0.5% or more, about 1.0% or more, about 2.5% or more, about 5% or more, about 10.0% or more, about 15.0% or more, about 25.0% or more, or about 30.0% or more up to about 45.0%, based on the weight of the oral care composition. In further embodiments, the sugar alcohol is present in an amount of 0.5% to 6%, about 10% to about 40%, about 15% to about 40%, about 20% to about 40%, or about 30% to about 40%, based on the weight of the oral care composition.

The compositions of the present invention may optionally comprise additional ingredients suitable for use in oral care compositions. Examples of such ingredients include, but are not limited to, flavoring agent, sweetener, abrasive, colorant, minerals, trace elements, vitamins, and additives.

In certain embodiments, the compositions may further comprise a flavoring agent. The flavoring agent may comprise one or more essential oils as well as various flavoring aldehydes, esters and/or alcohols. In certain embodiments, the flavoring agent comprises one or more essential oil selected form oils of peppermint, wintergreen, sassafras, clove, sage, eucalyptus, marjoram, cinnamon, lemon, lime, grapefruit and orange. In certain embodiments, the flavoring agent comprises oils of peppermint and spearmint. In certain embodiments the composition comprises from 0.05 to 3.0 weight %, 0.05 to 2.0 weight % or 0.08 to 2.0 weight % flavoring agent based on the total weight of the composition.

In any of the above embodiments, the compositions may further comprise a sweetener such as, for example, sodium saccharin. In certain embodiments, the sweetener may be included in various forms, such as but not limited to, a free base, a salt such as a hydrochloride (HCl) salt, or peptidyl forms. In one embodiment, the sweetener may be present in an amount of from about 0.2% to about 1.5%, based on the weight of the oral care composition. For example, the sweetener may be present in an amount of about 0.2 weight %, about 0.25 weight %, about 0.30 weight %, about 0.35 weight %, about 0.40 weight %, about 0.45 weight %, about 0.50 weight %, about 0.55 weight %, about 0.65 weight %, about 0.85 weight %, about 1.00 weight %, about 1.20 weight %, about 1.40 weight %, or about 1.50 weight %. In another example, the sweetener may be present in an amount of from about 0.2% to about 1.0%, from about 0.2% to about 0.8%, or about 0.2% to about 0.6%, based on the weight of the oral care composition. In certain embodiments, the sweetener is present in an amount of about 0.2% to about 0.8%, about 0.2% to about 0.6%, or about 0.2% to about 0.4%, based on the weight of the oral care composition.

In certain embodiments, the compositions may further comprise an abrasive. In certain embodiments, the abrasive may comprise a silica. In certain embodiments, the abrasive may comprise one or more silica. Non-limiting examples of silica abrasives include silica gels or precipitated amorphous silicas, hydrated silica, and silica dimethyl silate. In certain embodiments, the silica is hydrated. In certain embodiments, the silica is fumed. In certain embodiments, the silica is a combination of hydrated and fumed silica.

In another aspect, the present disclosure provides for a method of treatment or prevention of erosive tooth demineralization, gingivitis, plaque, and/or dental caries, the method comprising the application to the oral cavity of a person in need thereof an oral care composition as described herein.

In another embodiment, the invention encompasses a method to improve oral health comprising applying an effective amount of the oral composition of any of the embodiments set forth herein to the oral cavity of a subject in need thereof.

In a further embodiment, the disclosure provides a method for preparing an oral care mouth rinse comprising combining together a single source of stannous, wherein the single source of stannous is stannous oxalate; a tertiary amine fluoride; and at least one of a monocarboxylic acid, dicarboxylic, or a tricarboxylic acid. The method of preparation may include further combining one or more elements as are described herein.

In certain embodiments the ionic tin is present in the oral care mouth rinse composition in an amount (assuming complete dissolution of the tin salt) sufficient to provide tin ions, preferably stannous ions, in an amount of from 0.01% to 0.10% by weight of the oral care mouth rinse composition; or in an amount of from 0.02% to 0.08% by weight of the oral care mouth rinse composition; or in an amount of from 0.03% to 0.06% by weight of the oral care mouth rinse composition; or in an amount of from 0.035% to 0.045% by weight of the oral care mouth rinse composition.

In some embodiments, the compositions of the present disclosure contain polyvinylpyrrolidone (PVP), also known as polyvidone, povidone, and having a CAS Registry number of 9003-39-8. PVP is a water-soluble polymer made from the monomer N-vinylpyrrolidone which has been shown reduce tooth staining by various compounds including ionic tin, without compromising oral care efficacy. Polyvinylpyrrolidone can have a molar mass of from 2,500 to 3,000 ,000 Daltons. In certain embodiments the PVP in the compositions of the present disclosure has a molar mass of from 10,000 to 300,000 Daltons, for example 20,000 to 100,000 or 40,000 to 75,000 Daltons. In certain embodiments the PVP has a molar mass of about 58,000 Daltons. Non-limiting examples of such PVP polymer suitable for use in the compositions of the invention are sold by Ashland, Inc., Covington, Ky. under the name Plasdone K-29/32, Plasdone S-630, Plasdone K-90 USP.

Typically, the PVP is present in the oral care mouth rinse formulations of the present disclosure in an amount from about 0.1% to about 4%, or about 0.15% to about 3% by weight of the composition. In certain embodiments, the PVP is present in the in an amount from about 2% to about 4%, or about 2% to about 3%, by weight of the composition.

In some embodiments, the compositions of the present disclosure include at least one alpha-hydroxy acid or salt thereof. In some embodiments, the salt of the at least one .alpha.hydroxy acid is the sodium salt or the potassium salt. In some embodiments, the salt is the sodium salt. In some embodiments, the at least one .alpha.-hydroxy acid is a C₃ to C₇ alpha-hydroxy acid, or a C₄ to C₆ alpha-hydroxy acid. In certain embodiments, the at least one alpha.-hydroxy acid is malic acid, tartaric acid, alpha-hydroxy glutaric acid, gluconic acid, or a salt thereof. In certain embodiments, the at least one alpha-hydroxy acid or salt thereof is selected from malic acid and sodium-D-gluconate.

In some embodiments, the at least one alpha-hydroxy acid or salt thereof is malic acid. In certain embodiments, the malic acid is a racemic mixture of L-malic acid and D-malic acid. In some embodiments, the malic acid is present in an amount of from 0.01% to 0.5%; for example from 0.05% to 0.3%; for example from 0.1% to 0.2%, by weight of the composition.

In some embodiments, the compositions of the present disclosure include at least one surfactant or solubilizer. Suitable surfactants include neutral surfactants (such as ethoxylated and hydrogenated ethoxylated castor oils which correspond to a CAS Registry number of 61788-85-0 or fatty acids of sugars), cationic surfactants (such as the ammonium cation surfactants) or zwitterionic surfactants. These surfactants or solubilizers can be present in amounts of typically about 0.01% to about 2%, preferably about 1% to about 2%, by weight of the oral care composition. In some preferred embodiments, the compositions of the present disclosure include a surfactant selected from polyoxyethylene (polyethylene glycol; PEG) hydrogenated castor oil, alkyl trihydroxyethyl propylenediamine, and combinations thereof. In some such embodiments, the compositions of the present disclosure include a PEG-40 hydrogenated castor oil, in an amount of from about 0.01% to about 2%, or from about 0.05% to about 2%, or from about 0.1% to about 2%, or about 1.8%, by weight of the composition. Suitable PEG-40 hydrogenated castor oils include, as non-limiting examples, polyoxyl hydrogenated castor oil (e.g., polyoxyl 40 hydrogenated castor oil), or those sold by BASF under the name Cremophor®.

In any of the above embodiments, the compositions can further comprise one or more polyhydric alcohols such as xylitol, glycerine, sorbitol, propylene glycol and combinations thereof. In certain embodiments the compositions can optionally comprise from about 0.10% to about 10% polyhydric alcohol by weight of the composition. In certain embodiments the compositions can comprise xylitol in an amount of from 0.50% to 10%, for example from 0.50% to 7.0%, for example from 1% to 5%, for example 2.5% xylitol, by weight of the composition.

In any of the above embodiments, the compositions can further comprise one or more sweeteners such as, for example, saccharin, for example sodium saccharin, acesulfam, neotame, cyclamate, sucrose, dextrose, sucralose, thaumatin, stevioside, glycyrrhizin, sorbitol, xylitol, maltitol, mannitol, or a mixture thereof. The one or more such sweeteners can be present in a total amount of from 0.005% to 5% by weight, for example 0.01% to 1%, for example 0.01% to 0.1%, for example 0.06% by weight, of the composition.

One or more colorants can be included in the compositions of the present disclosure. Colorants can include pigments, dyes, and agents imparting a particular color or visual quality to the composition. Any orally acceptable colorant can be used. One or more colorants can optionally be present in the compositions in an amount of from 0.00001% to 2%, for example from 0.0001% to 1%, for example from 0.00015% to 0.7% of the composition by weight.

Humectants can reduce evaporation and also contribute towards preservation by lowering water activity, and can also impart desirable sweetness or flavor to compositions. Suitable humectants include edible polyhydric alcohols such as glycerin, sorbitol, xylitol, propylene glycol as well as other polyols and mixtures of these humectants. Other useful materials can also include orally acceptable alcohols, or polymers, e.g., such as polyvinylmethyl ether maleic acid copolymers, polysaccharides (e.g. cellulose derivatives, for example carboxymethyl cellulose, or polysaccharide gums, for example xanthan gum or carrageenan gum).

A wide variety of preservatives can be used in the compositions of the present disclosure. Suitable preservatives include, for example, sodium benzoate, potassium sorbate, methylisothiazolinone, paraben preservatives, for example methyl p-hydroxybenzoate, propyl p-hydroxybenzoate, and mixtures thereof.

The oral care compositions of the present disclosure can also include a flavoring agent. Flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is typically incorporated in the oral composition at a concentration of 0.01 to 1.5% by weight.

In some embodiments, the oral compositions of the present disclosure include antitartar agents to prevent and/or minimize calculus formation. One or more of such agents can be present. Suitable anticalculus agents include without limitation: stannous, copper, magnesium and strontium salts, dimethicone copolyols such as cetyl dimethicone copolyol, glucoamylase, glucose oxidase, urea, calcium lactate, calcium glycerophosphate, strontium polyacrylates and chelating agents such as citric and tartaric acids and alkali metal salts thereof, and salts of EDTA, for example tetrasodium EDTA; and phosphates and polyphosphates. Phosphate and polyphosphate salts are generally employed in the form of their wholly or partially neutralized water soluble cationic species (e.g., potassium, sodium or ammonium salts, and any mixtures thereof). Thus, useful inorganic phosphate and polyphosphate salts illustratively include monovalent cations with monobasic, dibasic and tribasic phosphates; tripolyphosphate and tetrapolyphosphate; mono-, di-, tri- and tetra-pyrophosphates; and cyclophosphates (also generally known in the art as “metaphosphates”). Useful monovalent cations of such phosphate salts include hydrogen, monovalent metals including alkali metals, and ammonium, for example.

In some embodiments, the oral compositions of the present invention may comprise one or more sensates, i.e., ingredients which impart some kind of sensation to the oral cavity. Suitable sensates include without limitation, physiological cooling agents including 1-menthol and 3-(1-menthoxy)propane-1,2-diol, peppermint oil, N-substituted-p-menthane-3-carboxamides, acyclic tertiary and secondary carboxamides, and 3-1-menthoxy propan-1,2-diol (see, e.g., PCT Published Application Number WO 97/06695); heating and/or warming sensates such as, for example and not imitated to, vanillyl alcohol n-butyl ether (vanillyl butyl ether), vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, isoamyl alcohol, benzyl alcohol, chloroform, eugenol, cinnamon oil, connamic aldehyde and phosphate derivatives of same; materials that are known to cause a tingling, numbing and/or stinging sensation and are used in foods as popular spice and/or herb condiments; and combinations thereof.

In some embodiments, the oral compositions of the present disclosure comprise one or more odor-neutralizing agents. Suitable odor neutralizing agents include, without limitation, chlorine dioxide; peroxides such a hydrogen peroxide; chlorite salts and bicarbonate salts,--e.g. sodium chlorite and sodium bicarbonate; essential oils such as eucalyptol, menthol, methyl salicylate and thymol; flavor cocktails; and zinc salts such as, for example and not limited to, zinc chloride, zinc citrate, zinc acetate, zinc sulfate, and zinc phenolsulfate.

The compositions of the present disclosure can comprise a saliva stimulating agent useful, for example, in amelioration of dry mouth. One or more saliva stimulating agents are optionally present in saliva stimulating effective total amount.

The compositions of the present disclosure can include antisensitivity agents. Such agents can be added in effective amounts, e.g., from about 0.1 wt. % to about 5 wt. % by weight based on the total weight of the composition, depending on the agent chosen.

In any of the above embodiments, the compositions can further comprise a pH adjuster. For example, the compositions can comprise an acid or base in an amount sufficient to adjust the pH of the compositions such that the compositions have a pH of from 3.0 to 8.0.

Water is present in the oral compositions of the invention. Water employed in the preparation of commercial oral compositions should be deionized and free of organic impurities. Water commonly makes up the balance of the compositions and includes 10% to 99%, e.g., 40% to 98.5%, e.g., 80% to 98.5%, e.g., 85% to 98% by weight of the oral compositions. This amount of water includes the free water which is added plus that amount which is introduced with other materials or any components of the invention.

As will be evident to one of skill in the art, some components of the present compositions may perform multiple functions, and the identification of a compound as having one function herein is not meant to exclude its use for other functions in a particular composition. For example, a compound such as xylitol can function in the compositions of the invention as a sweetener, but also act as a humectant.

In some embodiments, the invention is use of a composition according as described herein to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health.

In some embodiments, the compositions of the invention are intended to be used as a mouth rinse, delivering a dose of the ionic tin and fluoride to the oral cavity of a subject.

The present compositions are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

The present invention in its method aspect involves applying to the oral cavity a safe and effective amount of the compositions described herein. The compositions and methods according to the present disclosure are useful, inter alia, to cleanse and/or lubricate the oral cavity of a mammal, for example a human, and in particular to clean and/or lubricate the oral cavity and provide improved methods of promoting oral health and/or systemic health, including cardiovascular health, e.g., by reducing bacterial levels in the oral cavity.

Enhancing oral health also provides benefits in systemic health, as the oral tissues can be gateways for systemic infections. Good oral health is associated with systemic health, including cardiovascular health. The compositions and methods of the invention are thus useful to enhance systemic health, including cardiovascular health.

In certain embodiments, the present disclosure provides for a method for mitigating gut microbiome putrefaction in a companion animal, comprising feeding an effective amount of any of the compositions described above.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

Formulations of various mouth rinse compositions were prepared and evaluated. Table 1 shows the formulation of various samples. Amounts are provided as wt. %.

TABLE 1

| Ingredients | Sample 1 | Sample 2 | Sample 3 | Sample 4 |
|---|---|---|---|---|
| Tin (II) Oxalate | 0.071 | 0.071 | 0.071 | 0.0530 |
| Water | 95.26 | 95.47 | 97.46 | 97.46 |
| Xylitol | 0.8373 | 0.8373 | 0.8373 | 0.8373 |
| Water soluble PVP | 2.28 | 2.28 | 2.285 | 2.285 |
| Saccharin | 0.05 | 0.05 | 0.05 | 0.05 |
| Aroma | 0.11 | 0.11 | 0.11 | 0.11 |
| PEG-40 hydrogenated castor oil | 0.225 | 0.225 | 0.225 | 0.225 |
| Tertiary Amine fluoride | 0.93 | 0.93 | 0.93 | 0.93 |
| NaF | 0.028 | 0.028 | 0.028 | 0.028 |
| Tin (II) pyrophoshate | - | - | - | 0.018 |
| Pigment Blue | 0.0002 | 0.0002 | 0.0002 | 0.0002 |
| KOH | 0.069 | - | - | - |
| Malic acid | 0.1386 | - | - | 0.0069 |

As is shown in Table 1, samples 1 through 3 contained stannous oxalate as a stannous source. Sample 1 further contained a dicarboxylic acid. Sample 4 contained both stannous oxalate and stannous fluoride as a stannous source and also contained a dicarboxylic acid. The compositions of Table 1 were incubated at either 25° C. or 40° C. for up to six months and physical appearance visually assessed. The results are shown in Table 2 below.

TABLE 2

| | | | | |
|---|---|---|---|---|
| | Sample 1 | Sample 2 | Sample 3 | Sample 4 |
| Initial at 25° C. | Clear blue solution | Clear blue solution | Clear blue solution | Almost clear blue solution |
| 3 months at 25° C. | Clear blue solution | Moderately turbid solution | Moderately turbid solution | Turbid solution |
| 3 months at 40° C. | Clear blue solution | Turbid solution with sedimentation | Turbid solution with sedimentation | Turbid solution with sedimentation |
| 6 months at 30° C. | Clear blue solution | Turbidity | Turbidity | Turbidity |
| 6 months at 40° C. | Clear blue solution | Turbidity | Turbidity | Turbidity |

As shown in Table 2, Sample 1 maintained a stable clear blue solution for up to 6 months at temperatures up to 40° C. In contrast, Samples 2 to 4 became turbid under similar conditions. Significantly, sample 4, which contains stannous fluoride, quickly became turbid. Existence of turbidity is associated with a lack of galenic stability. Further, in consumer applications, maintaining a clear solution is aesthetically beneficial.

### Example 2

Biofilms of Streptococcus mutans were incubated for 24 hours and exposed to a rinse. The antimicrobial efficacy of mouth rinse was evaluated by use of isothermal microcalorimetry (IMC) of a 50% dilution of the original rinse solution. Each series of experiments included growth controls of untreated biofilms (no mouth rinse added) and a negative medium control (no mouth rinse and no biofilm added). Figure 1 shows the results of the experiments. Results are shown as the mean and standard deviation (i.e. mean +/- standard deviation).

TABLE 3

| Sample | Growth Rate (⅟h) | Lag Time | Avg. Reduction of Active Biofilms Compared to Control |
|---|---|---|---|
| Untreated Control | 0.078 +/- 0.008 | 8.90 +/- 0.80 | N/A |
| Commercial rinse containing stannous fluoride and tertiary amine fluoride | 0.075 +/- 0.006 | 19.52 +/- 2.15 | 56.3 |
| Sample 1 | 0.073 +/- 0.006 | 17.58 +/- 2.55 | 49.2 |

Table 3 shows the results of antimicrobial efficacy experiments. Sample 1 rinse showed an average efficacy of 49.2%, similar to commercial rinse, which utilized stannous fluoride as a stannous source, and which showed an average efficacy of 56.3%.

### Example 3: Effect of Trisodium Citrate Dihydrate

Formulations of various mouth rinse compositions were prepared and evaluated. Table 4 shows the formulation of various samples. Amounts are provided as wt. %.

TABLE 4

| Ingredients | Sample 5 | Sample 6 | Sample 7 |
|---|---|---|---|
| Sorbitol | 36.0 | 36.0 | 36.0 |
| Water | 26.52 | 27.49 | 25.49 |
| Hydrated Silica | 16.6 | 16.6 | 16.6 |
| Cocoamidopropyl betaine | 4.3 | 4.3 | 4.3 |
| Tertiary amine fluoride | 5.2 | 5.2 | - |
| Fumed silica | 2.7 | 2.7 | 2.7 |
| Tertiary amine fluoride and SnF₂ | - | - | 7.0 |
| Hydroxyethylcellulose | 2.2 | 2.2 | 2.2 |
| PEG-40 hydrogenated castor oil | 1.8 | 1.8 | 1.8 |
| Flavor | 1.6 | 1.6 | 1.6 |
| Sodium gluconate | 1.55 | - | 1.55 |
| Tin (II) oxalate | 0.61 | 0.61 | - |
| Saccharin | 0.385 | 0.385 | 0.385 |
| HCl (32%) | 0.30 | 0.38 | - |
| Trisodium citrate dihydrate | - | 0.5 | - |
| Sodium fluoride | 0.232 | 0.232 | - |
| 50% caustic potash | - | - | 0.378 |
| Pigment | 0.0015 | 0.0015 | 0.0015 |

The compositions of samples 5 through 7 were tested at various temperatures and relative humidity (RH). The amount of either stannous or fluoride in solution was then assessed. The results are shown in Table 5 below.

TABLE 5

| | | | Sample 5 | Sample 6 | Sample 7 |
|---|---|---|---|---|---|
| Sn (II) (ppm) | 30° C./65 % RH | 0 months | 2613 | 3045 | 1711 |
| | | 3 months | 1639 | 1436 | 760 |
| | | 6 months | 1286 | 1456 | 542 |
| | 40 °C/75 % RH | 3 months | 1409 | 1112 | 658 |
| | | 6 months | 621 | 1080 | 202 |
| Ionic Fluoride (ppm) | 30° C./65 % RH | 0 months | 1415 | 1413 | 1394 |
| | | 3 months | 1395 | 1365 | 1263 |
| | | 6 months | 1429 | 1268 | 1357 |
| | 40 °C/75 % RH | 3 months | 1435 | 1305 | 1180 |
| | | 6 months | 1165 | 1200 | 1239 |

Sample 5 and sample 6, which utilized stannous oxalate as the sole source of stannous, showed improved amounts of soluble stannous when compared to sample 7, which used stannous fluoride as a stannous source. Surprisingly, Sample 6, which contained sodium citrate, showed high Sn (II) content both initially and up to 6 months after incubation under accelerated aging conditions at 40° C.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. An oral care composition comprising:
a single source of stannous, wherein the single source of stannous comprises stannous oxalate;
a tertiary amine fluoride; and
at least one of a monocarboxylic acid, dicarboxylic, or a tricarboxylic acid.

2. The oral care composition according to claim 1, wherein the oral care composition is a mouth rinse.

3. The oral care composition according to claim 1, wherein the stannous oxalate is present at about 0.04% to about 3.0%, based on the weight of the oral care composition, the tertiary amine fluoride is present in an amount from about 0.7% to about 8.0% by weight, of the oral care composition, and the total amount of monocarboxylic acid, dicarboxylic, and tricarboxylic acid present is in an amount from about 0.1% to about 5.0% by weight, of the oral care composition.

4. (canceled)

5. The oral care composition according to claim 1, wherein the tertiary amine fluoride is olaflur.

6. The oral care composition according to claim 1, wherein the monocarboxylic acid, dicarboxylic, or a tricarboxylic acid is selected from gluconic acid, malic acid, citric acid, or combinations thereof.

7. (canceled)

8. The oral care composition according to claim 1, wherein the composition further comprises an amphoteric surfactant in an amount from about 2.0% to about 6.0% by weight, of the oral care composition.

9. The oral care composition according to claim 8, wherein the amphoteric surfactant comprises cocamidopropyl betaine.

10. (canceled)

11. The oral care composition according to claim 1, wherein the composition further comprises hydroxyethyl cellulose in an amount from about 1.0% to about 5.0% by weight, of the oral care composition.

12. (canceled)

13. The oral care composition according to claim 1, wherein the composition further comprises a sugar alcohol in an amount from about 0.5% to about 45.0% by weight, of the oral care composition.

14. The oral care composition according to claim 13, wherein the sugar alcohol comprises sorbitol, xylitol, or a combination thereof.

15. (canceled)

16. The oral care composition according to claim 1, wherein the composition further comprises saccharin in an amount from about 0.2% to about 1.0% by weight, of the oral care composition.

17. (canceled)

18. (canceled)

19. A mouth rinse composition comprising:
a single source of stannous, wherein the single source of stannous comprises stannous oxalate;
a tertiary amine fluoride;
hydroxyethylcellulose;
an amphoteric surfactant; and
at least one of a monocarboxylic acid, dicarboxylic, or a tricarboxylic acid.

20. The mouth rinse composition according to claim 19, wherein the stannous oxalate is present at about 0.04% to about 3.0%, based on the weight of the mouth rinse composition.

21. The mouth rinse composition according to claim 19 wherein the tertiary amine fluoride is present in an amount from about 0.7% to about 8.0% by weight, of the mouth rinse composition, the tertiary amine fluoride comprising olaflur.

22. (canceled)

23. The mouth rinse composition according to claim 19, wherein the monocarboxylic acid, dicarboxylic, or a tricarboxylic acid is in an amount from about 0.3% to about 5.0% by weight, of the mouth rinse composition, and is selected from gluconic acid, malic acid, citric acid, or combinations thereof.

24. (canceled)

25. The mouth rinse composition according to claim 19, wherein the amphoteric surfactant comprises cocamidopropyl betaine in an amount from about 2.0% to about 6.0% by weight, of the mouth rinse composition.

26. (canceled)

27. The mouth rinse composition according to claim 19, wherein the hydroxyethyl cellulose is present in an amount from about 1.0% to about 5.0% by weight, of the mouth rinse composition.

28. The mouth rinse composition according to claim 19, wherein the composition further comprises a sugar alcohol in an amount from about 0.5% to about 45.0% by weight, of the mouth rinse composition, the sugar alcohol comprising sorbitol, xylitol or a combination thereof.

29. (canceled)

30. (canceled)

31. The mouth rinse composition according to claim 19, wherein the composition further comprises saccharin in an amount from about 0.2% to about 1.0% by weight, of the mouth rinse composition.

32. (canceled)

33. (canceled)

34. A method of treatment or prevention of erosive tooth demineralization, gingivitis, plaque, and/or dental caries, the method comprising the application to the oral cavity of a person in need thereof an oral care composition comprising:
a single source of stannous, wherein the single source of stannous comprises stannous oxalate;
a tertiary amine fluoride;
at least one of a monocarboxylic acid, a dicarboxylic, or a tricarboxylic acid;
an amphoteric surfactant;
optionally, a hydroxyethyl cellulose in an amount from about 1.0% to about 5.0% by weight, of the oral care composition;
optionally, a sugar alcohol in an amount from about 0.5% to about 45.0% by weight, of the oral care composition;
optionally, saccharin in an amount from about 0.2% to about 1.0% by weight, of the oral care composition.

35-49. (canceled)
